(19) United States
(12) Patent Application Publication
(10) Pub. No.: US 2006/0276810 A1
(43) Pub. Date: Dec. 7, 2006

(54) Methods and apparatus for excising tissue and creating wall-to-wall adhesions from within an organ

(76) Inventors: Brian Kelleher, San Diego, CA (US); Christopher Paul Swain, London (GB)

Correspondence Address: SCOTT EVANS, 1252 COUNTRY HILLS DR., SANTA ANA, CA, 92705, US

(21) Appl. No.: 11/418,691

(22) Filed: May 6, 2006

Related U.S. Application Data
(60) Provisional application No. 60/679,284, filed on May 9, 2005.

(51) Int. Cl. A61B 17/08

(52) U.S. Cl. 606/151

(57) ABSTRACT

The preferred methods and devices described herein provide for creating adhesions between the tissue layers of folded soft tissue. The methods described can be accomplished with the use of an endoscope and once folds of tissue are formed and brought together using methods known in the art, the folds can be secured using a clamping system. This clamping system retains the tissue folds in a side by side relationship by exerting a clamping pressure against the folds. Over time the clamping pressure may initiate mucosal layer necrosis, deep tissue layer intermingling and tissue knitting.

### CLAIM OF PRIORITY

This application claims priority under 35 U.S.C. § 119(e) to U.S. Provisional Application No. 60/679,284, filed May 9, 2005, the entire contents of which are hereby expressly incorporated by reference.

### BACKGROUND

1. Field of the Invention

The present invention relates to devices and methods for joining segments of soft tissue together.

2. Description of the Related Art

Often segments of soft tissue are brought together for the purpose of resecting tissue, providing anchors for other devices and for creating walls or partitions within an organ having a lumen. Sometimes a single wall of tissue is folded and brought together and other times two portions of soft tissue are grasped separately and then the two portions are brought into close proximity to each other and then joined together either permanently or temporarily. The joining of portions of soft tissue has traditionally been done using clamping, banding, suturing or stapling devices. However, joining segments of tissue together whereby some of these segments may be exposed to tension post-operatively often does not hold up over time. For example, when two discrete segments of the stomach wall are sewn together the sutures that hold the segments together are in tension post-operatively. In order to prevent the sutures or other fastening devices from pulling through the stomach wall over time, the sites where the devices puncture the outer wall of the stomach are sometimes reinforced with sections of tear-resistant material called pledgets or other designs must be deployed to prevent pull out.

The placement of staples, sutures and the use of pledgets is not always possible especially when securing the wall of an organ that has a surface not easily accessible during the procedure. As an example, when performing an endoluminal gastroplasty procedure, that is, when sewing the wall of the stomach to itself from within the lumen of the stomach, only the inner wall of the stomach is accessible. Sutures that are placed through these walls can be strain-relieved with a pledget or similar device only along the inner surface of the wall, but not along the outer wall (unless a pledget or similar device is passed through the wall, which is generally not practical).

To solve this problem, at least one and often two folds of stomach wall are approximated and anchors are placed through the folds from inside the stomach. However these anchors need to be reinforced to avoid the same issues with pull out described earlier. One method of reinforcing these secured folds is to approximate the tissue folds and then anchor them together which can sometimes cause the tissue folds to weld or bond together thus creating a formed tissue bridge that is permanent. These tissue bridges are typically stronger than tissue clamped together without such tissue fusing.

U.S. Pat. application Ser. No. 2004/0215216 to Jamy Gannoe, the entirety of which is hereby included by reference, discloses a tissue approximation and fixation device. The device is used to approximate two folds of soft tissue to form a pleat to be used for gastric reduction surgery or GERD. In this application, Gannoe fixates portions of tissue together so that the tissue can fuse or scar over. In this application however Gannoe specifically discusses the need to apply a clamping force that does not clamp too tightly thus leading to complications such as pressure necrosis or too lightly which may result in an incomplete tissue union. Thus inconsistent securement is a problem that requires precise application of force. The present invention, as will be shown, does not require precise application of clamping force. The present invention describes a clamp and method that are designed to at least cause pressure necrosis in one portion of tissue leading to tissue knitting in adjacent portions of the tissue fold.

There is therefore a need for devices and methods that enable tissue-to-tissue securement with reduced chance of detachment occurring post-operatively. More specifically, there is a need for devices and methods that join tissue walls together, provide pressure on the joint and promote inner tissue layer intermingling. Additionally, these tissue securement devices need to be delivered endoscopically, as through a rigid endoscope, or endoluminally, as through a flexible endoscope.

### BRIEF SUMMARY OF THE INVENTION

The preferred methods and devices described herein provide for joining together the inner layers of an organ's tissue that has been folded. The methods described can be accomplished with the use of an endoscope and once folds of tissue are formed and brought together using methods known in the art, the folds can be secured using a clamping system. This clamping system retains a portion of the tissue walls in a side by side relationship by exerting a clamping force against these soft tissue folds. Over time the clamping force may initiate mucosal layer pressure necrosis which can lead to an erosion or dissipation of the mucosal layer. This action may promote the contact of other deep tissue layers such as the muscularis or serosa, which can lead to intermingling of cells and tissue knitting. The clamping device eventually works its way deeper into other layers of tissue and eventually migrates all the way through the adjoining tissue and falls off leaving behind a permanent healed tissue seam or bridge in other adjoining portions of the tissue folds. It is expected that the tissue seam formed may be stronger than alternative tissue joining methods because the resultant seam is similar to the natural stomach wall.

In one aspect of the present invention, a soft tissue fold clamping system comprises a spring clamp that is sized to be placed with the aid of an endoscope. The tissue clamping device comprises first and second arms that are joined at one end by a force member which biases the first and second arms together. The first arm terminates at its other end in a first clamping pad and likewise the second arm terminates at its other end in a second clamping pad. The force member imparts a force to the clamping pad that is sufficient to necrose tissue along at least one portion of the tissue fold.

The clamp is capable of exerting a steady and constant clamping force onto the soft tissue folds. The clamping device may be designed such that it is available with various spring forces and various initial or final diameters so that the operator can select the clamp with the amount of force or the size required for the thickness of the folded tissue.

In another aspect of the present invention, the clamp also includes tissue retention elements that are disposed about the open ends of the first and second clamping pads. These retention elements are designed to secure the ends of the clamp to the tissue and may be a barb, a serration, a hook, a tooth or teeth, or a roughened or tacky surface.

In another aspect of the present invention, the clamp also includes a cutting element that is located near the retention elements. The cutting element may be useful for gradually penetrating the tissue fold to facilitate severing the tissue fold once tissue necrosis has occurred. In still another aspect of the present invention, the clamp also includes a tethering element coupled to the clamp at one end. The tethering element is comprised of a cord with a tissue anchor coupled to the opposite end. The tissue anchor may be suitable for at least temporary coupling to a soft tissue wall at a location other than at the tissue fold. The tether may secure the clamp to the tissue wall so that the clamp can be retrieved later.

Another embodiment of the invention includes a method for joining together layers of folded tissue. The method includes forming a fold of tissue, applying a clamp to the fold of tissue with the clamp imparting a clamping force to the fold. The clamping force applied should be sufficient to necrose one portion of the folded tissue with the clamping force causing other portions of the tissue fold that are near the necrosed tissue to intermingle and form a tissue bridge with each other.

All of these embodiments are intended to be within the scope of the present invention herein disclosed. These and other embodiments of the present invention will become readily apparent to those skilled in the art from the following detailed description of he preferred embodiments having reference to the attached figures. The invention is not limited to any particular preferred embodiment(s) disclosed.

### BRIEF DESCRIPTION OF THE DRAWINGS

FIG. 1 is a section view showing a double fold of tissue and its layers of soft tissue.

FIG. 2 is a perspective view of a clamp positioned onto a double fold of soft tissue of FIG. 1.

FIG. 3 is a perspective view of the clamp of FIG. 2 showing transition zones of the clamped tissue.

FIG. 4a is a perspective view of the clamp of FIG. 2 with the clamp partially penetrating through the folds of soft tissue.

FIG. 4b is a perspective view of the clamp of FIG. 2 with the clamp more completely penetrating through the folds of soft tissue.

FIG. 4c is a perspective view of the clamp of FIG. 2 with the clamp completely penetrating through the folds of soft tissue.

FIG. 5 is a perspective view of a clamp coupled to a tissue wall with a tether.

FIG. 6 is a view of a stomach showing the arrangement of multiple clamp anchors.

FIG. 7 is a perspective view showing a fold of soft tissue after placement of a clamp anchor having a cutting element.

### DETAILED DESCRIPTION OF THE INVENTION

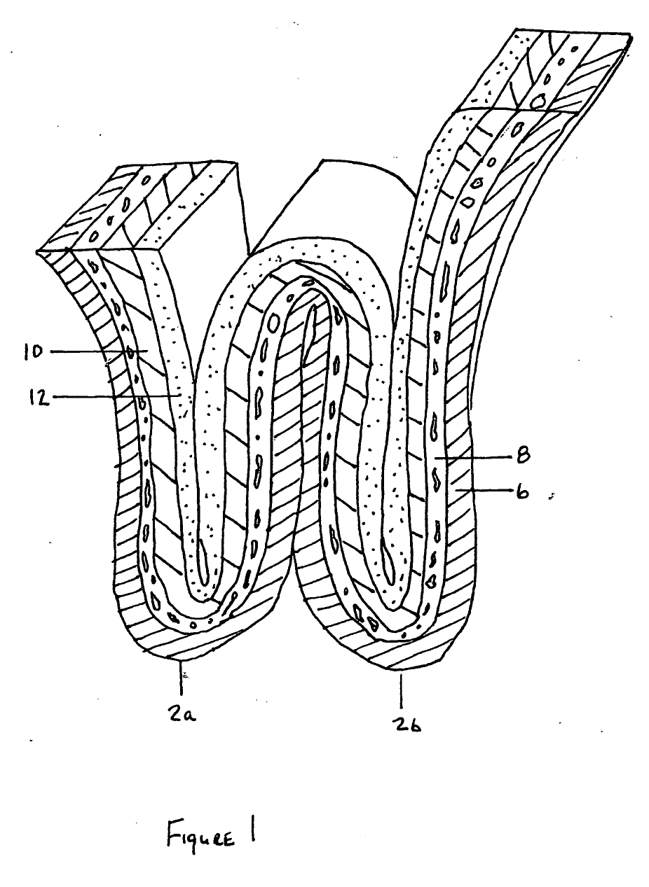

As has been described, folds of soft tissue are brought together and clamped for many reasons. As an example, folds of tissue are brought together for the purpose of reducing the volume of the stomach or other such organs. In the treatment of obesity, a gastric partition can be created in the stomach wall to restrict the intake of food to a smaller gastric volume. In this technique, the walls of the stomach are brought together using endoscopic techniques and secured together. When this procedure is performed using multiple sequential attachments, the volume of the stomach can be significantly reduced. In the first step of this procedure, the walls of the stomach are folded and usually two folds are brought into close proximity with each other as shown in FIG. 1. In this illustration, a section of a stomach wall 1 been formed by bringing together two folds 2a and 2b of stomach tissue together. The folds are brought together such that the inner surfaces of the stomach wall are brought into contact with each other. The stomach wall is composed of four primary layers; the mucosa 6 is the innermost layer, the submucosa 8, the muscle 10 and the serosa 12 or the outermost layer. The tissue layers shown in FIGS. 1 through 7 are meant to be an illustration only and are not necessarily anatomically correct. The thicknesses of various tissue layers may vary from that shown. If the folds of the soft tissue 2a and 2b are secured together with a fastening device, suture or clamp such that a clamping force is applied to the layers, eventually the soft mucosal layer 6 may erode exposing other layers of the stomach wall. Whereas the soft mucosal layer 6 has been shown to slough off, the inner layers of the stomach wall, the submucosa 8 and the muscle 10 may be more stable and may grow together and become knitted into a single tissue layer when they are forced together over a period of time. This new single tissue layer forms a new stomach wall that is thought to be inherently stronger than two layers secured together with only fastening devices and no such tissue ingrowth. Because this area of tissue knitting or joining is strong, there is a greater likelihood that any anchors placed into the joined tissue will remain in place even when a tension load is applied to these anchors.

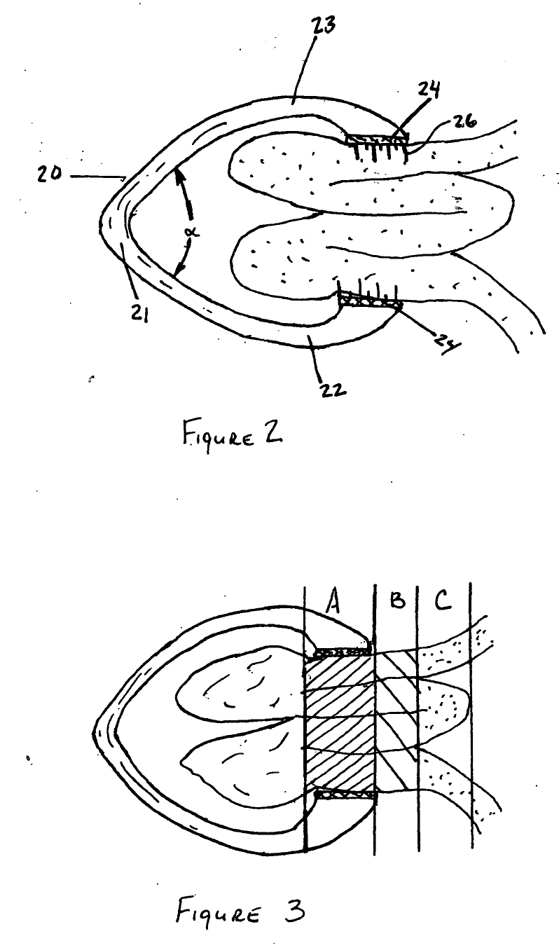

One embodiment of an improved wall to wall adherence device is the soft tissue clamp 20 shown in FIG. 2. The clamp 20 is deployed with the aid of an endoscope and is biased toward the closed state. The clamp is biased closed so that after it is spread open for deployment, the clamp will exert a clamping force on any tissue folds. After the stomach wall is folded as shown in FIG. 1, the endoscope (not shown) is brought adjacent to the folds 2a and 2b and the clamp 20 is moved from a closed state to an open state and positioned about the folded tissue. The clamp 20 is comprised of a force member 21, spring arms 22 and 23 and at least one clamping surface or pad 24. The force member 21 exerts a clamping force on the clamp arms 22 and 23 and on the clamping surface 24 such that the clamping pad 24 pinches the tissue walls of the folded tissue together.

The spring force for this clamping system should be enough so that tissue necrosis is facilitated over time and the clamp does not fall off the tissue fold, There is no need to limit the maximum clamping force or confine the clamping force to a certain narrow range. One potential advantage of this type of clamp is that the range of forces available to necrose tissue and yet keep the clamp in contact with the fold is very broad and this may present an advantage over other tissue joining methods that require precise clamping forces be applied. Other methods and devices require that the clamping force be sufficient to pinch the tissue folds together but not enough to necrose tissue. The necrosis of tissue in other systems is described as something that should be avoided and is described as a possible adverse side effect of such a procedure.

However, often it is not precisely known preoperatively what the optimal clamping force should be. This is particularly difficult for several reasons. First is that the size and shape of the fold is variable. The endoscopic techniques and devices for fold formation are not precise and the resultant fold length, width and thickness will vary from operator to operator and from patient to patient. As the thickness and width of a fold vary, the clamping force required of the clamping system must vary as well to provide a uniform clamping force at the inner tissue layers. Secondly the biological makeup of the tissue itself is variable between patients and between folding sites. The density of the tissue, the muscle content and the degree of vascularization can vary greatly. In order to avoid tissue necrosis as taught by other clamping devices, which is caused when blood vessels are blocked leading to ischemic cell death, the clamping force needs to be less than any force that will obstruct blood vessels and halt blood flow. However a highly vascularized tissue will require a different clamping force to avoid blocking blood vessels than a poorly vascularized tissue bed. These variables cannot be predicted pre-operatively so it is very difficult to provide a precise clamping force.

One aspect of the current invention is that the clamping force does not need this precision. A clamp with a large clamping force can be designed that will provide adequate clamping force independent of the patient anatomy or the fold dimensions. The clamping force provided by the force member can be expressed as a force or as a force per unit area which is pressure. In one embodiment, the force member imparts a minimum force of 1 pound over one square inch, exerting a pressure of one pound per square inch (psi). In another more preferred embodiment the force member exerts a minimum pressure of 5 psi. And in a most preferred embodiment the force member exerts a pressure in the range of 2 to 20 psi.

The force member 21 may be integrally formed with the arms 22 and 23. In this case the force member 21 may be constructed of metal, plastics, shape memory alloys or composites or combinations of all of these materials. The force member may also be a separate element that is coupled to the inner apex of arms 22 and 23. Examples of this type of force member 21 may be a coil or leaf spring (not shown). Although a “c-type” clamp is illustrated in FIG. 2, the tissue joining methods described are not dependent on a particular type or structure of clamp. Many clamp designs and many clamping mechanisms are known in the art that are suitable for endoscopic use and which provide the necessary clamping force required to necrose tissue and fuse one tissue wall with another tissue wall.

The clamp as shown in FIG. 2 has an angle at between the two arms 22 and 23. This angle is the smallest when the clamp is in it's closed, resting state. The angle is increased as the clamp 20 is opened prior to placement onto the folded tissue. Once in position on the tissue, the force member 21 causes the arms to move together and the angle decreases as the clamping force is applied to the folds. As will be shown the angle may gradually continue to decrease over time as the clamping surface or pads 24 move closer toward each other. This movement is caused by the gradual migration of the pads through the tissue is a result of the spring force exerted on the soft tissue. The clamping force causes a reduction of blood delivery to the tissue as blood vessels are pinched closed. Without blood delivery nutrients are not supplied to the cells and waste products are not removed. Eventually, this ischemic condition leads to tissue necroses, and the tissue is broken down and cells are not regenerated. The bulk of the tissue walls will begin to break down. As this occurs, the pads 24 will gradually move closer together.

The pads 24 may include a retention element 26 that is designed to insure that the clamp 20 does not prematurely detach from the tissue wall. This is helpful to insure that the clamp 20 remains in position until the intermingling of inner tissue walls has taken place. The retention element 26 may be a modified surface on the pads 24. Examples of this are for example a roughened surface, a serrated surface or even a tacky or sticky surface, all of which may improve the fixation of the pad 24 to the tissue. The pad 24 may also utilize barbs, hooks, teeth or other mechanical fixating members that can penetrate into inner tissue layers.

The effect of the clamp 20 on the folded tissue varies depending on the proximity of the tissue to the clamp pad 24. As shown in FIG. 3, three zones of tissue interaction are illustrated, each having a different clamping force and with resultant different cellular reaction to the applied force. Zone A is the zone directly between clamping pads 24. This zone receives the largest clamping force, and substantially all of the blood supply to this region of tissue is cut off by the applied force. Zone B is adjacent to zone A and receives a smaller clamping force than zone A. Zone C is adjacent to zone B and receives a smaller clamping force than zone A or zone B. The tissue in zone A should receive enough pressure from the clamp 20 such that pressure necrosis is initiated in this zone throughout the full thickness of the clamped section of tissue, including soft mucosal layer 6, sub mucosa 8, muscle 10 and the serosa 12.

The tissue located in adjacent zone B receives less pressure than zone A but the pressure received by zone B may still be enough to cause a reduction in blood supply, leading to necrosis and erosion of at least the soft mucosal layer. However the outer tissue layers, the muscularis and the serosa, in this zone may not fully necrose but rather may intermingle and fuse together over time. Almost immediately after application of clamp 20 to the folded tissue, zone B will become inflamed as a reaction to the injury taking place to the adjacent tissue in zone A. The ensuing inflammatory response is thought to contribute to the process which leads to fusion of tissue in zone B. This union of the tissues from one fold to another may result in a new tissue wall having similar structural properties of native tissue. The tissue of zone C receives such a small resultant clamping force that little or no tissue necrosis or tissue erosion occurs in this area. The tissue in this zone remains relatively untouched by the folding and clamping process.

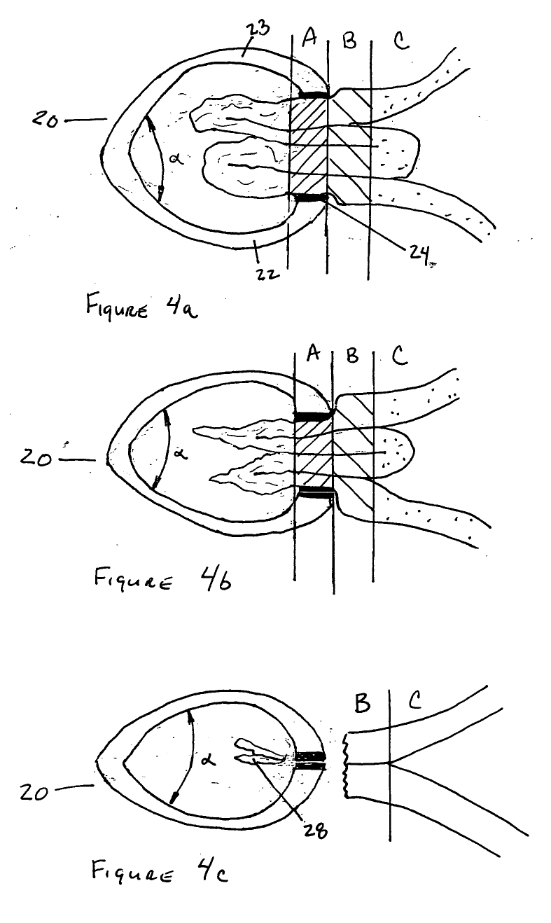

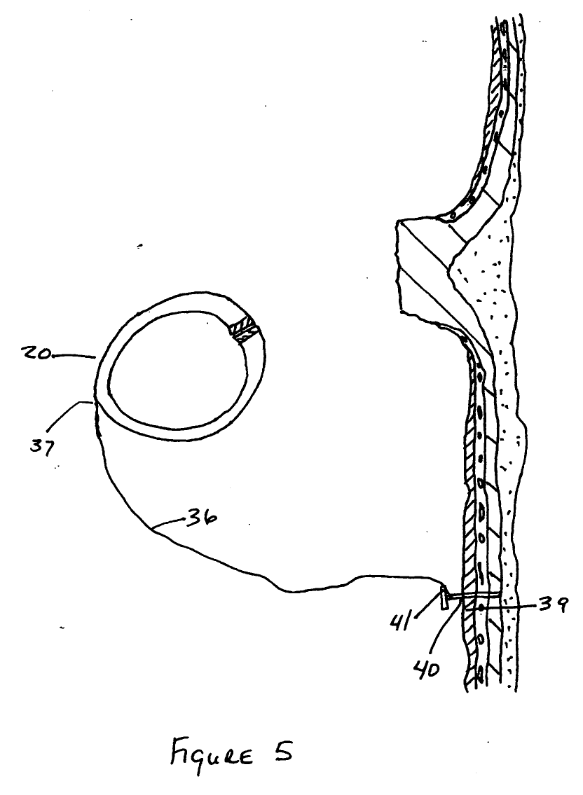

The process of tissue erosion and fusion may take place over hours or days or even weeks and in different zones as has been described. In some areas of tissue contact, all layers of the tissue wall may necrose completely and be sloughed off by the body; in other areas where the clamping force is less, only portions of the tissue layer will necrose and the other layers will intermingle and fuse; and in still other areas the clamping pressure is insufficient for any necrosis to occur and in this area no tissue intermingling will occur. The zones illustrated in FIG. 3, are not exact and the actual width of the zones may vary. In FIG. 4a, the clamp 20 is shown after continual clamping pressure on the tissue has initiated partial pressure necrosis of the tissue in zone A. The angle a between the two clamp arms 22 and 23 as shown in FIG. 4a is less than the initial clamping angle a shown in FIG. 3 and each pad 24 is closer to each other than in the initial deployment condition. As shown in FIG. 4b, each pad 24 has more completely migrated through the tissue wall in zone A. However, in zone B which is the zone adjacent to zone A, only some of the soft tissue layers have necrosed and the submucosa and muscle layers can intermingle and form new combined tissue. In zone c, adjacent to zone B, where the resultant clamping force is insufficient to cause any tissue interaction, no intermingling has occurred.

Eventually the tissue in zone A may necrose and erode to the point that it separates completely from the tissue of zone B as illustrated in FIG. 4c. When this occurs, the clamp 20 with the necrosed tissue portion 28 separates from the other tissue. The tissue of zone B however, remains as new combined tissue and should provide a secure wall to wall attachment. The tissue of zone C remains essentially unaffected.

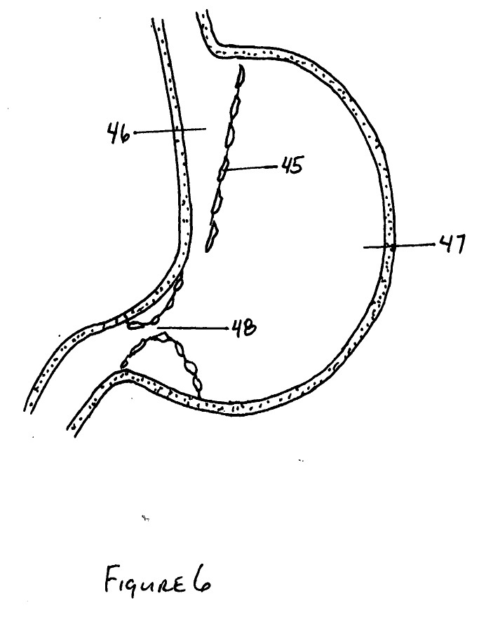

As the clamp is released into the body organ, and by example the stomach, the clamp and tissue may pass through the stomach and intestines and be expelled from the body. This may be possible because the size of the clamp 20 and folds illustrated are small and these may pass through the digestive tract without sequelae. Alternatively, the clamp may be constructed from bioabsorbable materials that dissolve over time or are reabsorbed by the body. And finally the clamp 20 may utilize a tether 36 as shown in FIG. 5 that attaches to the clamp 20 at some point 37 and the tissue wall 39 at point 40. The point 40 is located at a position other than the site of the tissue folds and clamping. The tether 36 may also have an anchor 41 at the opposite end that is secured to the tissue wall 39. This tether 36 holds the clamp 20 in the organ and prevents any migration of the clamp until the clamp 20 can be removed using endoscopic techniques.

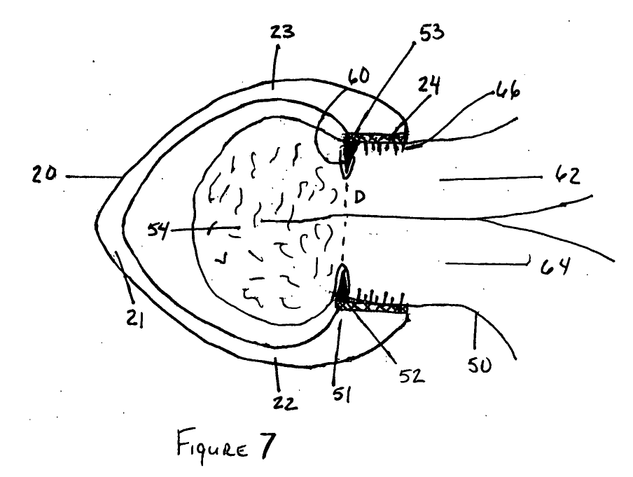

In another embodiment of the invention, one or more clamps such as clamp 20 could be used to form a line of attachments 45 as shown in FIG. 6. A string of clamps could be used to form a gastric pouch 46 in stomach 47. Additionally another string of attachments could form a restrictive gastric outlet 48. These clamps could be placed in close proximity to each other so as to form essentially a continuous wall that effectively partitions the stomach into portions with reduced volumes.

In another embodiment of the invention the clamp 20, may be used to resect and clamp one or more folds or tucks of soft tissue. In this embodiment, shown in FIG. 7, the clamp 20 is a spring clamp with two arms 22 and 23 and with the pad 24 at the each end of the arms. However the clamp 20 can be used to secure one or more folds of tissue 50 in such organs as the bowel, stomach, pancreas, liver, gall bladder and their related ducts. The pad 24 may also have at least one cutting element that can cut a portion of tissue while the clamp holds other portions of the tissue together. A clamp of this type may be useful when trying to remove damaged tissue 54 such as cancerous polyps, tumors or other growths or as a method to remove tissue volume. The cutting element removes tissue while the clamp keeps the cut ends together to reduce bleeding and to promote healing and tissue intermingling. As shown, the cutting element 51 has two cutters; a lower cutter 52 and an upper cutter 53. However a cutting element with only one cutter or more than two cutters is possible. Likewise, other cutting mechanisms such as serrated ridges, sharpened blades, knife edges and other tissue cutters known in the art are anticipated. In FIG. 7, the cutting element 51 has partially severed the tissue at point 60 and the pads 24 are holding the two ends 62 and 64 of the tissue fold 50 together. The clamp remains attached to the tissue until it is sloughed off as described previously. This clamp is particularly useful to occlude blood supply to the clamped tissue to prevent initial bleeding. As the two clamp arms 22 and 23 continue to gradually migrate together, the cutters 52 and 53 gradually cut through the tissue along line D. Eventually, the cutters meet together along the clamp centerline and the damaged or diseased tissue is severed completely and is passed through the intestines and out of the body. This embodiment of the invention may also utilize a retention element 66 that is similar to retention element 26 as previously described.

Although this invention has been disclosed in the context of certain preferred embodiments and examples, it will be understood by those skilled in the art that the present invention extends beyond the specifically disclosed embodiments and/or uses of the invention and obvious modifications and equivalents thereof Thus it is intended that the scope of the present invention herein should not be limited by the particular disclosed embodiments described above, but should be determined only by a fair reading of the claims that follow.

What is claimed is:

1. A tissue clamping device comprising: first and second arms joined at one end by a force member, the force member biasing the clamp in a closed position, said first arm terminating at its other end in a first clamping surface and said second arm terminating at its other end in a second clamping surface, the force member imparting a force to the clamping surface which exerts sufficient pressure to necrose tissue along at least one portion of at least one fold of tissue.

2. The device of claim 1 wherein the exerted pressure is greater than 1 psi.

3. The device of claim 1 wherein the exerted pressure is greater than 5 psi.

4. The device of claim 1 wherein the exerted pressure is sufficient to cause tissue intermingling at other portions of the tissue fold that are disposed about the necrosed tissue.

5. The device of claim 4 further comprising a first and second tissue retention element disposed about the first and second clamping surface.

6. The retention element of claim 5 wherein the retention element is a barb, a serration, a hook, a tooth or teeth, or a roughened or tacky surface.

7. The force member of claim 1 wherein the force member and the arms are formed from a single piece of metal or plastic.

8. The clamp of claim 4 further comprising a tethering element coupled to the clamp at one end, the tethering element comprising a cord and having an anchor coupled to the opposite end, the anchor suitable for coupling to tissue at a location other than at the tissue fold.

9. The tethering element of claim 8 wherein the tethering element tethers the clamp to tissue and prevents distal migration of the clamp.

10. The device of claim 1 further comprising a cutting element adjoining at least one of the first or second clamping surfaces, said cutting element sized to gradually penetrate and sever the at least one tissue fold.

11. A method of joining together layers of at least one fold of tissue comprising:
forming the fold of tissue, applying a clamp to the at least one fold of tissue, the clamp exerting
a pressure to the fold that is sufficient to necrose one portion of the tissue.

12. The method of claim 11 wherein the exerted pressure causes other portions of the tissue fold that are disposed about the necrosed tissue to intermingle and form a tissue bridge with each other.

13. The method of claim 12 wherein the tissue is part of a hollow body organ.

14. The method of claim 13 wherein the hollow body organ is a stomach.

15. The method of claim 13 wherein the clamp is delivered with an endoscope to the hollow body organ.

16. The method of claim 11 further comprising two folds of tissue wherein the tissue has a mucosal layer, the mucosal layer of the two tissue folds adjoin, and the clamping force causing the mucosal layer of a portion of the tissue folds that are disposed about the necrosed tissue to dissipate leaving other tissue layers adjacent to each other.

17. The method of claim 16 wherein the other tissue layers are submucosa, muscularis or serosa.

18. The method of claim 16 wherein the other layers adjacent to each other intermingle and form a tissue bridge with each other.

19. The method of claim 16 wherein the clamp and the necrosed tissue portion separate from the portions of the tissue fold that are disposed about the necrosed tissue.

20. The method of claim 19 wherein the clamp further comprises a tethering element that is tethered to a portion of a tissue wall that is spaced apart from the fold.

21. The method of claim 20 wherein the tethering element comprises a cord and an anchor, the anchor suitable for at least temporarily coupling to the tissue wall at a location other than at the tissue fold.

22. The method of claim 11 wherein the clamp further comprises a cutting element and the cutting element severs a damaged or necrosed portion of the tissue fold from adjacent portions.

23. A method of joining together layers of at least two folds of tissue inside the body comprising:
forming and approximating the folds of tissue, applying a clamp to the at least two folds of tissue, the clamp exerting a pressure to the folds that is sufficient to necrose one portion of the at least two folds of tissue.

24. The method of claim 23 wherein the tissue has a mucosal layer, the mucosal layer of the two tissue folds adjoin, and the exerted pressure causing the mucosal layer of a portion of the tissue folds that are disposed about the necrosed tissue to dissipate leaving other tissue layers adjacent to each other.

25. The method of claim 24 wherein the other tissue layers are submucosa, muscularis or serosa.

26. The method of claim 24 wherein the other layers adjacent to each other intermingle and form a tissue bridge with each other.

27. The method of claim 26 wherein the tissue is part of a hollow body organ.

28. The method of claim 27 wherein the hollow body organ is a stomach.

29. The method of claim 26 wherein the clamp is delivered with an endoscope to the hollow body organ.

30. The method of claim 26 wherein the clamp and the necrosed tissue portion separate from the portions of the tissue fold that are disposed about the necrosed tissue.

31. The method of claim 30 wherein the clamp is tethered to a portion of the tissue that is spaced apart from the folds of tissue.

32. The method of claim 31 wherein the clamp further comprises a tethering element that is coupled to the clamp at one end.

33. The method of claim 32 wherein the tethering element comprises a cord and an anchor, the anchor suitable for at least temporarily coupling to a tissue wall.

34. The method of claim 26 wherein the clamp further comprises a cutting element and the cutting element severs a damaged or necrosed portion of the tissue fold from adjacent portions.
